(19) United States
(12) Patent Application Publication
(10) Pub. No.: US 2017/0231650 A1
(43) Pub. Date: Aug. 17, 2017

(54) Endoscope Attachable Cystolith Entrapment Apparatus

(71) Applicant: Peter Sayet, Fort Lauderdale, FL (US)

(72) Inventor: Peter Sayet, Fort Lauderdale, FL (US)

(21) Appl. No.: 15/435,095

(22) Filed: Feb. 16, 2017

Related U.S. Application Data
(60) Provisional application No. 62/295,712, filed on Feb. 16, 2016.

(51) Int. Cl. A61B 17/22; A61B 1/018

(52) U.S. Cl. CPC A61B 17/22031; A61B 1/018; A61B 2017/0034

(57) ABSTRACT

An endoscope attachable cystolith entrapment apparatus, attachable to an extant endoscope, includes a sleeve mount and an operative end. A plurality of distensible leaves, actionable at the operative end, is moveable between a closed position, furled and retracted interior to an anterior aperture disposed at the operative end, and an open position, unfurled and extended out from the anterior aperture by manual action at a deployment member. Each of the plurality of distensible leaves is transparent whereby vesical calculi are sightable, targetable, and thence captuarable within a capture volume during cystoscopy, and thence removable from a bodily lumen absent direct contacting with adjacent tissue.

### BACKGROUND OF THE INVENTION

Vesical calculi can form anywhere in the lower urinary tract, become deposited in the bladder, and gain significant size; procedures such as lithotripsy, cystolitholapaxy, and other reductive procedures, become necessary to reduce the targeted calculus to smaller sized particles for extraction or expulsion through the urethra. In some conditions, open cystostomy may be required. These procedures are invasive, expensive, and potentially destructive to host tissue.

The present practice of lassoing calculi, and any daughter stones created during reduction, is difficult at best and, if successful, often allows for persistent contact between the calculus and epithelial tissue lining the lumen through which the stone is subsequently extracted. This can damage urothelial cells, aggravate existing conditions, and create inflammation and discomfort for the patient.

Additionally, cystoscopy is often performed first, to visualize the calculus previous to extraction procedures. In some instances multiple insertions are required into the patient's bladder (or other cavity) to locate, reduce, capture, and extract the stone(s).

What is needed is a means of enabling enveloped capture of a stone when visualized during cystoscopy in general, whereby location of a stone immediately enables extraction of the stone visualized, and removal of the stone is enabled even when the stone's diameter (within limits) exceeds the diameter of the urethra of the patient in question.

### FIELD OF THE INVENTION

The present invention relates to an endoscope attachable cystolith entrapment apparatus devised to releasably secure endwise to an existing cystoscope and thereby enable enveloped capture of a visualized stone during the act of cystoscopy without having to repeat procedures to effect extraction after visualization and location of the stone have occurred. Additionally, due to the present device's attachability to existing cystoscopes, a user need only purchase the present apparatus for use with existing equipment already extant in surgery, equipment with which said user may have already become expert.

The present invention, then, includes a sleeve mount disposed to releasably secure to the distal end of an endoscope and thus position an operative end having plurality of distensible leaves endwise over said endoscope distal end. Each of the plurality of distensible leaves is transparent wherein visualization through the endoscope distal end remains unimpaired. Each of the plurality of distensible leaves is moveable from a closed position, retracted and furled within an anterior aperture disposed at the operative end, and an open position, extended and unfurled from without the anterior aperture.

A deployment member is disposed in operational communication with the operative end, and positioned proximally upon the endoscope when the device is secured thereto, whereby manual engagement of the deployment member during cystoscopy effects movement of the plurality of distensible leaves between the closed and open positions.

Each of the plurality of distensible leaves is disposed to envelop a calculus once visualized by an operator, and subsequently retain said calculus interiorly secured for extraction through the urethra. An external surface of each of the plurality of distensible leaves enables smooth contact with urothelial tissue and enables even relatively large calculi (with diameters exceeding the diameter of the urethra) to be extracted without open cystostomy required.

Thus the present invention enables installment of an operative end to pre-existing equipment whereby an endoscope (and particularly a cystoscope) is transformable into an instrument able to effect immediate capture and extraction of a calculus, or reduced calculi, whenever initially discovered in a patient's bladder.

### SUMMARY OF THE INVENTION

The general purpose of the endoscope attachable cystolith entrapment apparatus, described subsequently in greater detail, is to provide an endoscope attachable cystolith entrapment apparatus which has many novel features that result in an endoscope attachable cystolith entrapment apparatus which is not anticipated, rendered obvious, suggested, or even implied by prior art, either alone or in combination thereof.

The present endoscope attachable cystolith entrapment apparatus enables transformation of any endoscope into a device able to immediately capture vesical calculi upon visualization and obviates the need for repeated procedures.

The term “cystolith”, “vesical calculus”, or “calculus”, in either singular or plural form (“calculi”), is taken to mean any stone formable interior to the urinary and renal tracts, in addition to any hardened substance disconnected from any tissue desirously removable via extraction through a bodily lumen. While attention is given specifically to “cystoliths” and extraction from the bladder, it should be recognized by anyone having skill in the art that the same apparatus set forth herein could readily be employed at various scales, as required, to reach into the ureter and enable capture of nephroliths, for example, as well. Extraction of other objects from alternate lumens is likewise conceivable. The present device, then, is considerable for use really anytime extraction of a hardened substance, disconnected from endothelial or epithelial tissue on the whole, is warranted from any bodily lumen or cavity.

The terms “endoscope” and “cystoscope” are used interchangeably herein throughout, and are taken according to the ordinary meanings of the terms—a cystoscope being generally considered a specific type of endoscope particularly employed for conducting cystoscopy interior a patient's bladder. However, the term “endoscope”, as used herein, in concert with the term “cystoscope” throughout, implies broader applications of the present invention, and is set forth herein whereby no limitation of the device specifically for cystoscopy or any particular endoscopic procedure is intended, the general metes and bounds of the invention enabling visualization and enveloped capture of a target for infraluminal extraction.

The present endoscope attachable cystolith entrapment apparatus, therefore, includes a sleeve mount disposed to effect securable attachment to an endoscope. The sleeve mount is devised to accommodate existing models of endoscopes and may be adaptable to accommodate particular makes and models, as desired. Importantly, the sleeve mount enables securable attachment to an endoscope and positions an operative end distally thereto. A deployment member is likewise disposed proximally wherein an operator is enabled manual engagement of the deployment member for deployment of a plurality of distensible leaves, as will be described subsequently, while operating the endoscope.

Mounting the sleeve mount to an endoscope, therefore, disposes a plurality of distensible leaves endwise over the endoscope distal end. Each of the plurality of distensible leaves includes an arcuate body, a hooked tip, an external surface, an interior surface, and a pair of edges. The plurality of distensible leaves is moveable between a closed position, retracted and furled interior to an anterior aperture disposed endwise at the operative end, and an open position, extended and unfurled from out said anterior aperture.

When disposed in the closed position, each of the plurality of distensible leaves engages together and each hooked tip seats together to form an anterior node apically disposed upon the apparatus. Each of the plurality of distensible leaves is transparent, whereby visualization through the anterior node is unimpaired.

When moved to the open position, the plurality of distensible leaves is first projected forward, out the anterior aperture before splaying apart to define a capture volume. Retraction of the plurality of distensible leaves effects rejoining of each of the leaves together, sealably contacted along each edge, and effects reseating of the hooked tips to form the anterior node.

Thus, subsequent visualization of a calculus, an operator is enabled orientation of the operative end to target the calculus within the capture volume, deployment of the plurality of distensible leaves to the open position, entrapment of the calculus when retracting the plurality of distensible leaves toward the closed position, whereby the calculus is enveloped within the plurality of distensible leaves. Infraluminal extraction of the calculus is now enabled, and contact of the calculus directly with urothelial tissue is avoidable.

The external surface of each of the plurality of distensible leaves is contemplated to be smooth, ductile and glossed, whereby abrasion is minimized and fluid contact with surrounding tissue accommodated. The interior surface of each of the plurality of distensible leaves may be agglutinated to assist capture and retention of the targeted calculus, and may include an osmotic potential, or other diffusion potential, whereby the calculus, or any component part, is drawn and/or sorbed to said interior surface. Said interior surface may also include a reducing agent—that is, any agent chemically capable of altering, or reducing the size of, the stone once contact is effected therewith.

Due to the membranous quality of the plurality of leaves, and the glossed, smooth, external surface, relatively large calculi are considered extractable through the urethra without damage or aggravation thereto. The plurality of distensible leaves is configured to envelop the targeted calculus and enable extraction even at diameters exceeding that of the urethra (or other lumen, as case may be) as the external surface enables fluid contact with epithelial tissue.

Thus has been broadly outlined the more important features of the present endoscope attachable cystolith entrapment apparatus so that the detailed description thereof that follows may be better understood and in order that the present contribution to the art may be better appreciated.

For better understanding of the endoscope attachable cystolith entrapment apparatus, its operating advantages and specific objects attained by its uses, refer to the accompanying drawings and description.

### BRIEF DESCRIPTION OF THE DRAWINGS

### Figures

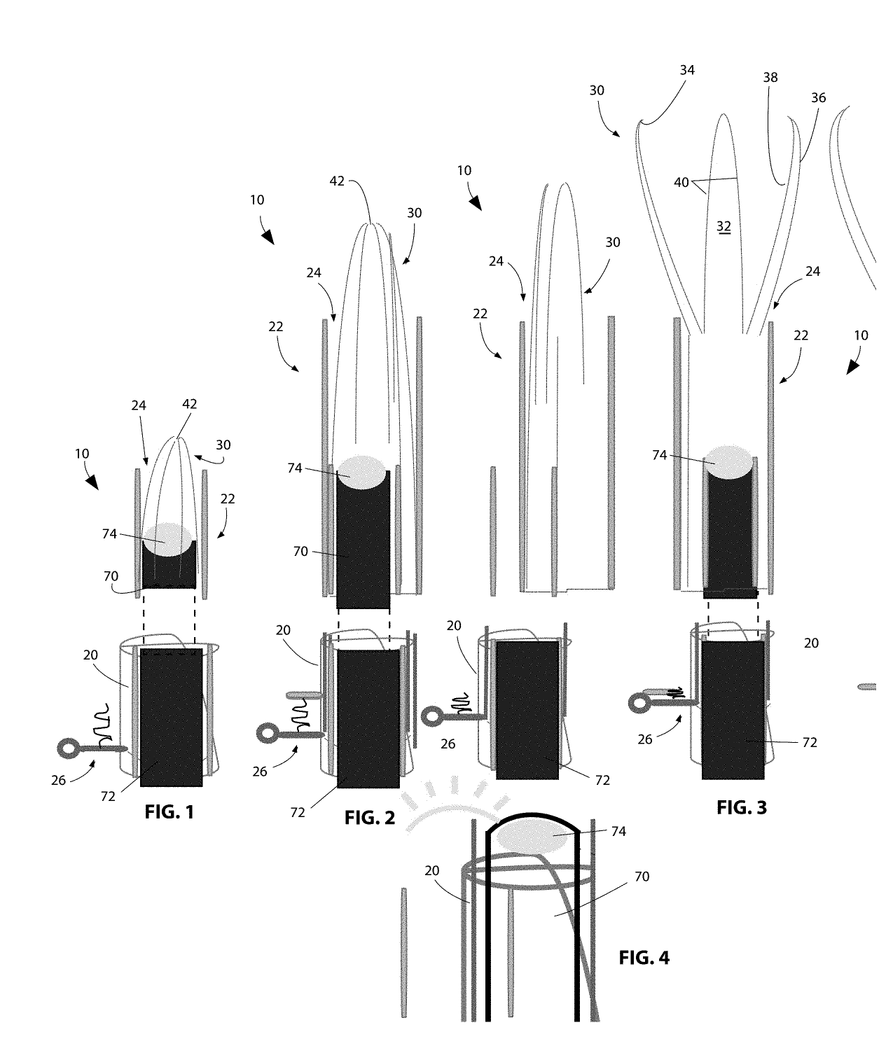

FIG. 1 is a plan view of an example embodiment of the apparatus in a closed position with a plurality of distensible leaves retracted and furled.

FIG. 2 is a plan view of an example embodiment of the apparatus in between a closed position and an open position.

FIG. 3 is a plan view of an example embodiment of the apparatus in an open position with the plurality of distensible leaves extended and unfurled.

FIG. 4 is a plan view of a sleeve mount attachable overtop a proximal end of a cystoscope.

### DETAILED DESCRIPTION OF THE DRAWINGS

With reference now to the drawings, and in particular FIGS. 1 through 4 thereof, example of the instant endoscope attachable cystolith entrapment apparatus employing the principles and concepts of the present endoscope attachable cystolith entrapment apparatus and generally designated by the reference number 10 will be described.

Referring to FIGS. 1 through 4 a preferred embodiment of the endoscope attachable cystolith entrapment apparatus 10 is illustrated.

The present endoscope attachable cystolith entrapment apparatus 10 has been devised to enable endwise securement to an extant cystoscope 70 for urethral insertion and introduction into the bladder to thereby snare vesical calculi for removal through the urethra during cytoscopy. The present endoscope attachable cystolith entrapment apparatus 10 is removably securable to a cystoscope 70 for use thereupon, and includes a plurality of distensible leaves 30 moveable between a closed position, retracted into an operative end 22 of the apparatus 10, and an open position, unfurled extended from out said operative end 22. Each of the plurality of distensible leaves 30 is transparent enabling user visualization therethrough when operating the extant cystoscope to which the present apparatus 10 is securably mounted.

The present endoscope attachable cystolith entrapment apparatus 10, therefore, includes a sleeve mount 20 disposed to releasably secure over an endoscope 70 (particularly a cystoscope) and secure an operative end 22 immediately overtop the endoscope 70 distal end 74. The operative end 22 includes an anterior aperture 24 through which the plurality of distensible leaves 30 is projectable. Each of the plurality of distensible leaves 30 is a transparent, membranous member preferably formed of polymeric material such as, for example, silicone or another hypoallergenic material. Importantly, visualization through the endoscope 70 is not significantly impaired by addition of the present apparatus 10, and any illuminable member extant upon or through the endoscope 70 is not impeded by opacity of the plurality of distensible leaves 30.

Each of the plurality of distensible leaves 30 includes an arcuate body 32, a hooked tip 34, an external surface 38, an interior surface 40, and a pair of edges 42 disposed to sealably engage in contact with each of a pair of neighboring distensible leaves, whenever the plurality of distensible leaves 30 is withdrawn to the closed position. Each of the hooked tips 34 is disposed to seat together to form an anterior node 42 apically bounding the apparatus 10 when the plurality of distensible leaves 30 is disposed in the closed position. The external surface 36 of each of the plurality of distensible leaves 30 is configured to be smooth, ductile, and potentially glossed whereby contact with urothelial tissue is fluid and discomfort to the patient is preventable. The interior surface 38 may be agglutinative and include an osmotic potential or other diffusion gradient potential to effect sorption, adhesion, or frictive engagement with a targeted calculus. A reductive agent—such that chemically alters the calculus in proximity therewith—may also be included.

The plurality of distensible leaves 30 is disposed in operational communication with a deployment member 26 disposed operatively connected to the sleeve mount 20 in a position appropriate for manual engagement by an operator during cystoscopy at a proximal end 72 of the endoscope 70. The deployment member 26 is engageable to effect movement of the plurality of distensible leaves 30 between the open and closed position.

Deployment of the plurality of distensible leaves 30 to the open position first extends the plurality of distensible leaves 30 out the anterior aperture 24, wherein each of the plurality of distensible leaves 30 separates from contact with each other and unfurls, the hooked tips 34 separating and spreading apart, whereby a catch volume is defined therebetween. Orientation of the operative end 22 to sight a vesical calculus, for example, thence enables deployment of the plurality of distensible leaves 30 from the closed position to the open position, whereby the calculus may be drawn into the catch volume and snared by action of returning the plurality of distensible leaves 30 to the closed position. The calculus is thence retained interior to the plurality of distensible leaves 30, which membranous property enables accommodation of the calculus while shielding the urothelial tissue during retraction of the endoscope 70.

Vesical calculi are therefore capturable and removable during cystoscopy in a single procedure without the need for the operator to perform visualization first, and then subsequently effect the removal in a separate step effective only with a change of equipment, as is currently seen in the art.

What is claimed is:

1. An endoscope attachable cystolith entrapment apparatus comprising;
a sleeve mount attachable to an extant endoscope;
an operative end distally disposed upon the sleeve mount;
an anterior aperture disposed at the operative end;
a plurality of distensible leaves actionable at the operative end, each of said plurality of moveable between a closed position, furled and to retracted interior to the anterior aperture, and an open position, unfurled and extended out from the anterior aperture; and
a deployment member disposed in operational communication with the plurality of distensible leaves;
wherein the plurality of distensible leaves is moveable between the closed position and the open position by action of the deployment member, whereby calculi are targetable, capturable, and removable from a bodily lumen.

2. The endoscope attachable cystolith entrapment apparatus of claim 1, wherein each of the plurality of distensible leaves is transparent.

3. The endoscope attachable cystolith entrapment apparatus of claim 2, wherein each of the plurality of distensible leaves comprises;
an arcuate body;
an external surface disposed exteriorly upon the arcuate body;
an interior surface disposed internally upon the arcuate body; and
a hooked tip disposed apically terminating the arcuate body;
wherein the hooked tip of each of the plurality of distensible leaves seats together to form an anterior node bounding the operative end when the plurality of distensible leaves is moved to the closed position.

4. The endoscope attachable cystolith entrapment apparatus of claim 3, wherein to the external surface of each of the plurality of distensible leaves is smooth and ductile whereby contact with urothelial tissue is fluid.

5. The endoscope attachable cystolith entrapment apparatus of claim 4, wherein the interior surface of each of the plurality of distensible leaves is agglutinative to effect sorption of proximally located calculi thereto.

6. The endoscope attachable cystolith entrapment apparatus of claim 5, wherein the interior surface of the plurality of distensible leaves includes a reductive agent capable of chemically altering and reducing calculi upon contact therewith.

7. An endoscope attachable cystolith entrapment apparatus comprising;
a sleeve mount attachable to an extant endoscope;
an operative end disposed distally upon the sleeve mount;
an anterior aperture disposed at the operative end;
a plurality of distensible leaves actionable at the operative end, each of the plurality of distensible leaves moveable between a closed position, furled and retracted interior to the anterior aperture, and an open position, unfurled and extended out from the anterior aperture, each of said distensible leaves comprising;
a transparent, arcuate body;
a smooth external surface disposed exteriorly upon the arcuate body;
an agglutinative interior surface disposed internally upon the arcuate body;
a hooked tip disposed apically terminating the arcuate body;
a pair of edges disposed to sealably engage in contact with each of neighboring pair distensible leaves; and
a deployment member disposed in operational communication with the plurality of distensible leaves, said deployment member manually engagable by a user operating the endoscope to effect movement of the plurality of distensibile leaves between the open and closed positions;
wherein the operative end is operable to sight a vesical calculus and deployment of the plurality of distensible leaves to the open position presents a catch volume for ensnarement of the calculus therein when the plurality of distensible leaves is returned to the closed position, each of the plurality of distensible leaves encapsulating the calculus, whereby removal of the calculus is effective without directly contacting adjacent unroethial cells.
